(19) United States
(12) Patent Application Publication
(10) Pub. No.: US 2009/0117255 A1
(43) Pub. Date: May 7, 2009

(54) Wheat Lines And Improved Food Compositions

(75) Inventor: Dirk B. Hays, College Station, TX (US)

Correspondence Address: Peter G. Carroll;MEDLEN & CARROLL, LLP, Suite 350, 101 Howard Street, San Francisco, CA, 94105, US

(73) Assignee: The Texas A&M University System

(21) Appl. No.: 12/208,916

(22) Filed: Sep. 11, 2008

Related U.S. Application Data
(60) Provisional application No. 60/993,362, filed on Sep. 12, 2007.

(51) Int. Cl. A21D 13/02; A01H 5/00; A21D 13/00; A21D 10/00

(52) U.S. Cl. 426/622; 800/320.3

(57) ABSTRACT

The invention relates to wheat lines and improved food compositions. In preferred embodiments, the invention relates to flour made from a wheat grain comprising genetic material with a null allele at one or more loci encoding a protein selected from the group consisting of glutenins and gliadins. In even more preferred embodiments, the invention relates to a tortilla made from said flour.

### FIELD OF INVENTION

The invention relates to wheat lines and improved food compositions. In preferred embodiments, the invention relates to flour made from a wheat grain comprising genetic material with a null allele at one or more loci encoding a protein selected from the group consisting of glutenins and gliadins. In even more preferred embodiments, the invention relates to a tortilla made from said flour.

### BACKGROUND

Hard red winter wheat (HRWW), the major wheat class grown in Texas and across the Southern Great Plains, has protein levels and gluten strength suitable for bread making. While wheat gluten functionality is also important for tortilla quality, most hard red winter wheat cultivars produce poor quality tortillas. Consumers usually prefer tortillas that exhibit acceptable appearance, taste and texture. However, since tortillas are not always consumed on the day they are baked, shelf stability is an important issue. Thus, there is a need to make tortillas with preferred consumer quality attributes while also maintaining extended shelf stability.

### SUMMARY OF THE INVENTION

The invention relates to wheat lines and improved food compositions. In preferred embodiments, the invention relates to flour made from a wheat grain comprising genetic material with a null allele at one or more loci encoding a protein selected from the group consisting of glutenins and gliadins. In even more preferred embodiments, the invention relates to a tortilla made from said flour.

In some embodiments, the invention relates to a composition produced from crushing a wheat grain or portion of a wheat grain, wherein a portion of said grain does not express a wild-type gene product from one or more loci encoding a protein selected from the group consisting of glutenins and gliadins. In further embodiments, said composition is flour. In further embodiments, said wheat grain does not express said gene product due to a deletion of a chromosome arm, deletion of said gene, or deletion of a portion of said gene at said loci. In further embodiments, said wheat does not express said gene product due to a mutation of said gene at said loci. In further embodiments, said gene product is selected from the group consisting of a protein or RNA. In further embodiments, said loci are glutenin loci selected from the group consisting of GluA1, GluB1, GluD1, GliA1, GliB1, GliD1, and Gli2A. In further embodiments, said wheat grain is obtained from a wheat line that is hexaploid and near-isogenic.

In some embodiments, the invention relates to a dough produced from a flour disclosed herein wherein said dough has a decreased elasticity compared to dough derived from a wheat line that does express said gene product from said loci.

In some embodiments, the invention relates to a flat bread produced from dough disclosed herein. In further embodiments, said flat bread is selected from the group consisting of tortilla, pizza dough, and pita.

In some embodiments, the invention relates to a wheat line comprising a null allele at a GluB1 locus. In some embodiments, the wheat line further comprises a null allele at a GluA1 locus. In some embodiments, the wheat line further comprises a GluA1 gene that expresses a protein subunit selected form the group consisting of subunit 1 and subunit 2*. In some embodiments, the wheat line further comprises a null allele at a GluD1 locus. In some embodiments, the wheat line further comprises one or more GluD1 genes that express a protein subunit selected from the group consisting of subunit 2, subunit 3, subunit 4, subunit 5, subunit 10, subunit 11, and subunit 12. In some embodiments, the wheat line further comprises a GluD1 gene that express protein subunits selected from the group consisting of subunits 5 and 10, subunits 2 and 12, subunits 3 and 12, subunits 4 and 12, subunits 2 and 11. In further embodiments, said wheat line does not express a gene product from one or more loci selected from the group consisting of GliA1, GliB1, GliD1 and Gli2.

In some embodiments, the invention relates to a wheat line comprising a null allele at a GluB1 locus, a GluA1 gene that expresses a protein subunit 1, and GluD1 gene that express protein subunits 5 and 10.

In some embodiments, the invention relates to a wheat line comprising a null allele at a GluA1 locus and GluB1 gene expressing a protein subunit selected form the group consisting of subunit 6, subunit 7, subunit 8, subunit 9, subunit 13, subunit 14, subunit 15, subunit 16, subunit 17, subunit 19, subunit 20, subunit 21, and subunit 22. In some embodiments, the wheat line further comprises a GluB1 gene expressing protein subunits selected from the group consisting of subunits 6 and 8, subunits 7 and 8, subunits 7 and 9, subunits 14 and 15, and subunits 17 and 18. In some embodiments, the wheat line further comprises a null allele at a GluD1 locus. In some embodiments, the wheat line further comprises a GluD1 gene expressing a protein subunit selected from the group consisting of subunit 2, subunit 3, subunit 4, subunit 5, subunit 10 and subunit 12. In some embodiments, the wheat line comprises a GluD1 gene expressing protein subunits selected from the group consisting of subunits 5 and 10, subunits 2 and 12, subunits 3 and 12, subunits 4 and 12, and subunits 2 and 11. In further embodiments, said wheat line does not express a gene product from one or more loci selected from the group consisting of GliA1, GliB1, GliD1 and Gli2.

In some embodiments, the invention relates to a wheat line comprising a null allele at a GluA1 locus, a GluB1 gene expressing protein subunits 17 and 18, and a GluD1 gene expressing subunits 2 and 12.

In some embodiments, the invention relates to a method comprising: a) providing a wheat line comprising a wheat grain wherein a portion of said grain comprises genetic material that does not express a wild-type gene product from one or more genes encoding a protein selected from the group consisting of glutenins and gliadins; and b) separating a grain from said wheat. In some embodiments, the method further comprises the step of processing said grain into a component selected from the group consisting of flour, meal, bran and grits.

In some embodiments, the invention relates to a method comprising: a) providing a wheat line that does not express a wild-type gene product from one or more gene loci selected from the group consisting of GluA1, GluB1, GluD1, GliA1, GliB1, GliD1 and Gli2; and b) milling a grain of said wheat under conditions to form a flour; c) mixing said flour with food ingredients to form a mixture and d) heating said mixture to form a food. In further embodiments, said mixture is a dough or batter. In further embodiments, said food is selected from the group consisting of bread, pasta, cracker, cereal, cake, gravy, sauce, soufflé, soup and stew. In further embodiments, said bread is a tortilla, bun, bunloaf, chapati, cholla, pita, potato bread, naan, and flat bread. In further embodiments, said heating is done by a method selected from the group consisting of baking, steaming, frying, broiling, roasting and grilling. In further embodiments, said heating is by an open flame, oven or hot surface. In further embodiments, said food ingredients include at least two components selected from the group consisting of non-wheat flour, water, salt, tapioca, sugar, spice, fruit, vegetable, nut, seed and a leavening agent. In further embodiments, said non-wheat flour comprises particles selected from the group consisting of maize, rye, barley, rice, grasses, buckwheat, grain amaranths, acacia, chestnut, chickpea, legumes, teff, lovegrass, peas, beans and nuts. In further embodiments, said flour is white flour, whole grain or germ flour. In further embodiments, said flour is bleached or bromated flour. In further embodiments, said milling comprises the steps of finely grounding wheat and endosperm of a grain of said wheat and coarsely grounding a bran and germ of a grain of said wheat. In further embodiments, said leavening agent is selected from the group consisting of yeast and backing soda.

In some embodiments, said flour has an ash mass of between 0.3 and 0.6 g per 100 g dry flour as determined by ICC Standard No. 104/1. In further embodiments, said flour has an ash mass of between 0.6 and 0.8 g per 100 g dry flour as determined by ICC Standard No. 104/1. In further embodiments, said flour has an ash mass of between 0.8 and 1.0 g per 100 g dry flour as determined by ICC Standard No. 104/1. In further embodiments, said flour has an ash mass of between 1.0 and 1.5 g per 100 g dry flour as determined by ICC Standard No. 104/1. In further embodiments, said flour has an ash mass of 1.5 g ash or more per 100 g dry flour as determined by ICC Standard No. 104/1. In further embodiments, said flour has a crude protein content of between 8-10% by weight as determined by ICC Standard No. 105/2. In further embodiments, said flour has a total protein content of between 10-12% by weight as determined by ICC Standard No. 105/2. In further embodiments, said flour has a total protein content of between 12-14% by weight as determined by ICC Standard No. 105/2. In further embodiments, said flour has a total protein content of greater than 15% by weight as determined by ICC Standard No. 105/2. In further embodiments, said flour has an ash mass of 1.5 g ash or more per 100 g dry flour as determined by ICC Standard No. 104/1 and has a total protein content of between 12-14% by weight as determined by ICC Standard No. 105/2.

In some embodiments, the invention relates to a method comprising: a) providing a wheat line comprising genetic material that does not express a wild-type gene product from loci selected from the group consisting of GluA1, GluB1, GluD1, GliA1, GliB1, GliD1 and Gli2; b) milling a grain of said wheat line forming a flour; and c) mixing said flour with a fat under conditions to form a roux. In some embodiments, the method further comprises the steps of d) mixing said roux with food ingredients to form a mixture and e) heating said mixture.

In some embodiments, the invention relates to malting and brewing. In some embodiments, it is contemplated that wheat grains from wheat lines disclosed herein are used to make flour for fermentation to make beer, alcohol, vodka or biofuel.

In other embodiments, wheat lines and grains disclosed herein are a forage crop for livestock, and the straw can be used as fodder for livestock or as a construction material for roofing thatch.

In some embodiments, the invention relates to wheat line comprising genetic material with mutations that alter the amino acid sequences of gliadins, preferably sequences disclosed herein. In even more preferred embodiments, these wheat lines express preferred glutenin gene products disclosed herein. In even more preferred embodiments, these wheat lines produce seeds that are crushed and used in food products.

### BRIEF DESCRIPTION OF THE DRAWINGS

FIG. 1 shows wheat line embodiments with protein compositions of parents and the HMW glutenin deletion lines. Deletions in a wheat line are indicated by a (-) in the table.

FIG. 2 shows data on % insoluble polymeric protein (IPP), % polymeric protein (PPP), dough development time (MDDT) and peak resistance (MU) from flour of wheat deletion lines grown in Texas (TX) for embodiments of the invention. For the HMW-GS near isogenic lines, deletions in a line are indicated by (-) in the chart.

FIG. 3 shows data of Pearson's correlations of % IPP and % PPP with dough and tortilla quality parameters developed from near-isogenic deletion lines grown in Texas.

FIG. 4 shows data of Pearson's correlations of % IPP and % PPP with dough and tortilla quality parameters developed from near-isogenic deletion lines grown in South Dakota.

FIG. 5 shows data on tortilla quality parameters of tortillas prepared from flour of wheat deletion lines grown in Texas (TX). Letters indicate Tukey's LSD significant difference groups.

FIG. 6 shows data on tortilla quality parameters of tortillas prepared from flour of wheat deletion lines grown in South Dakota. Letters indicate Tukey's LSD significant difference groups.

FIG. 7 illustrates contrasts between the glutenin alleles absent in the glutenin deletion lines and parent cultivars.

FIG. 8 shows calculations of dough and tortilla quality of wheat lines grown in Texas.

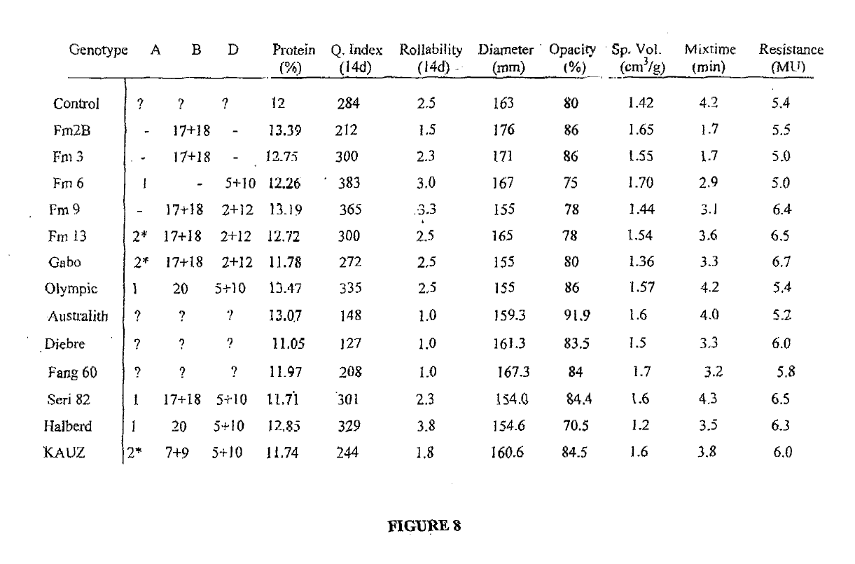

FIG. 9 shows calculations of dough and tortilla quality evaluations of the gliadin deletion lines grown in South Dakota.

### DETAILED DESCRIPTION OF THE INVENTION

When wheat flour is mixed with water, a complex protein called gluten develops. The gluten development is believed to give wheat dough an elastic structure that allows it to be worked in a variety of ways, and which allows the retention of gas bubbles in an intact structure, resulting in a sponge-like texture to the final product. This is highly desired for breads, cakes and other baked products. However, certain individuals suffer from an intolerance to wheat gluten known as coeliac or celiac disease. Increased awareness of this disorder, as well as a rising belief in the benefits of a gluten-free diet for persons suffering certain other conditions, has led to an increased demand for bread, pasta, and other products made with flours that do not contain gluten. Thus, in some embodiments, the invention relates to flour that has desirable elastic attributes made from wheat that can be tolerated by subjects, such as humans and animals, or subjects at risk for, diagnosed with or exhibiting symptoms of intolerance to wheat gluten.

Gluten is composed of high molecular weight (HMW), low molecular weight (LMW) glutenins (GS) and gliadins and their allelic variants. Wheat has three genomes (AABBDD) and it can encode for many variations of the same protein. Even in the gliadin subcategories, many types of gliadin exist per cultivar. For the following discussion of glutenin and gliadin variants, X designates a particular genome (A, B, or D genome) followed by the chromosomal number (1 to 7). Glutenins and gliadins on the Chromosome 1 short arm include ω-gliadin—(GliX1—A is null about 84%, B (>8 alleles), D (>4 alleles)), glutenin, LMW—(GluX3—A (>5 alleles), B (>7 alleles), D (>2 alleles)), γ-gliadins, (GliX3), and β-gliadins. Glutenins on the Chromosome 1 long arm include glutenin and HMW (GluX1—A (>2 alleles) B (>8 alleles) and D (>4 alleles)). For Chromosome 6 (A, B and D genomes), the short arm (˜30 coding loci over A, B, D undeterminant alleles) includes α-gliadin (GliX2), β-gliadins, (GliX2), and γ-gliadins (GliX2). Genetic studies indicate that, in wheat, each protein type can be encoded by several loci and several different alleles for each loci can be found in different genomes, allowing a great number of uniquely encoded isoforms.

### Glutenins

Glutenin is believed to be responsible for the firmness of dough in baking bread because it increases the stability through a three-dimensional network. HMW (High-Molecular-Weight) subunits are relatively low in sulfur. The LMW glutenins are encoded by the GluA3, GluB3 and GluD3 loci on the short arm of Chromosomes 1A, 1B and 1D, respectively.

The HMW-GSs are located on the long arm of Chromosomes 1A, 1B, and 1D. They are further subdivided into allelic pairs (x and y) on 1B and 1D and a single subunit on 1A. The allelic pairs encoded at the GluD1 locus (5+10, 2+12), the single subunit at the GluA1 (1, 2*, nil), and those encoded at the GluB1 locus (20, 7+9, 17+18) are believed to contribute to bread quality.

As used herein, a glutenin “subunit” refers to those disclosed in Payne et al., Cereal. Res. Commun. 11, 29-35 (1983); Payne et al., Philosophical Transactions of the Royal Society of London 304, 359-371 (1984), both of which are hereby incorporated by reference, mentioned as protein subunits that contribute to bread-making quality. Payne et al., Journal of the Science of Food and Agriculture 40, 51-65 (1987), incorporated by reference, describe over 50 wheat line varieties with various subunits and evaluations on bread characteristics. Wheat containing the GluA1 subunit 1 and GluD1 subunits 5 and 10 provide a good quality score. GluB1 subunit is preferably the 7+9 subunit. Shewry et al., Advances in Food and Nutrition Research 45, 219-302 (2003), hereby incorporated by reference, provide that subunits encoded by GluA1, GluB1 and GluD1 may be associated with quality and that the presence of GluA1 subunit 1 or 2* is better than the null allele.

Near-isogenic wheat lines can be developed to transfer a single gene or loci through backcrossing into a common genetic background. The affects of the individual gliadin and glutenin alleles or allelic pairs on dough and bread making quality can be studied without confusing the effects of different genetic backgrounds. Lawrence et al., J. Cereal Sci. 7, 109-112 (1988), incorporated herein by reference, disclose near-isogenic deletion wheat lines. These lines have been used to deduce effects on bread dough functionality as described in MacRitchie et al., Cereal Chem. 78, 501-506 (2001), hereby incorporated by reference.

As used herein, “wheat” refers to the grass (Triticum spp.). Some wheat species are diploid, e.g., einkorn wheat (T. monococcum), but many are stable polyploids, tetraploid, e.g., emmer and durum wheat derived from wild emmer, T. dicoccoides (wild emmer) is the result of a hybridization between two diploid wild grasses, T. urartu and a wild goatgrass such as Aegilops searsii or Ae. speltoides, or hexaploid, domesticated emmer or durum wheat hybridized with yet another wild diploid grass (Aegilops tauschii).

As used herein, a “null allele” refers to a deletion of all or a portion of a gene or a mutant of a gene that substantially changes the gene's normal function. This can be the result of the complete absence of the gene product (protein or RNA) at the molecular level, or the expression of a non-functional gene product, such as a truncated protein or RNA. A mutant allele that produces no protein is called a protein null, and one that produces no RNA is called an RNA null.

As used herein, “near-isogenic” wheat lines refer to strains of wheat genetically alike with respect to specified gene pairs. It is not intended to require that the entire genome be identical.

As used herein, a “wheat grain” refers to a seed of the wheat. The seed functions for reproducing, but the term is not intended to require that the seed be capable of reproducing. The germ is an embryo. The pericarp is a tough skin. The endosperm is the food reserve.

As used herein “crushing” refers to the pressing, grinding, pounding, and/or milling of an item into smaller particles, a powder, or a paste. The crushing of cereal grains (wheat, corn, rye, buckwheat, rice) into flour is an example of the use of tools to reduce particle size. The flour can then be eaten raw, cooked with water into porridge, or moistened, formed into a loaf, and baked as bread. Another nutritional advantage of the flour over the whole grain is that the flour can be sifted to remove the bran fraction, which is largely indigestible cellulose. The germ fraction of the kernel may be removed with the bran. Flours, rather than whole grains, also have the advantage of cooking faster and can be used to make gruels.

As used herein, “flour” refers to wheat flour obtained by crushing wheat grains or parts thereof into a powder or fine dust, e.g., wholemeal flour and white flour. In wholemeal flour, all parts of the grain are included, but in producing white flour the seed coats and the embryo are not used. Instead, they are flattened and removed as small flakes. These flakes are referred to as wheatfeed. It is not necessary that the powder or dust contain all of the original composition of the wheat grains. The term is intended to include non-enriched and enriched flour, i.e., flour with specific nutrients returned to it that have been lost while it was prepared. In preferred embodiments, the flour has, at minimum per pound, the following quantities of nutrients: 2.9 milligrams of thiamin, 1.8 milligrams of riboflavin, 24 milligrams of niacin, 0.7 milligrams of folic acid, and 20 milligrams of iron. Calcium also may be preferrably added at a minimum of 960 milligrams per pound.

In wheat (Triticum aestivum L) the synthesis of high molecular weight (HMW) glutenins (GS) is controlled by three heterologous genetic loci present on the long arms of group 1 wheat chromosomes. The loci GluA1, GluB1, GluD1 and their allelic variants play roles in the functional properties of wheat flour. In some embodiments, the invention relates to the functional aspects of tortilla quality made from flour from wheat lines where one or more of these loci do not express all the protein subunits. Near-isogenic wheat lines are contemplated in which one or more of these loci are altered, absent or deleted.

Tortillas were prepared from each deletion line and the parent lines. The elimination of certain HMW-GS alleles alters aspects of tortilla quality such as diameter, shelf stability and overall quality. Two deletion lines possessing HMW-GS 17+18 at GluB1 and deletions in GluA1 and GluD1 had significantly larger tortilla diameters, yet tortilla shelf life was compromised or unchanged from the parent lines used to develop the deletion lines or the commercial tortilla flour used as a control. Alternatively, a deletion line possessing GluA1 and GluD1 (HMW-GS 1, 5+10) and a deletion in GluB1 significantly improved tortilla diameters. While the increase in diameter was less than the line possessing only HMW-GS 17+18 at GluB1, the stability of the tortillas were maintained and improved compared to the parent lines containing a full compliment of HMW-GS. The presence of subunits 5+10 at GluD1 alone or in combination with subunit 1 at GluA1 appears to provide a compromise of improvement in dough extensibility for improved tortilla diameters while also providing sufficient gluten strength to maintain ideal shelf stability.

Tortillas about 2 mm thick, evenly opaque, with and ample diameter, and at least a three-week shelf life are preferred. As in bread, wheat flour and gluten functionality contributes to this shelf stability and the need for tortillas to resist breaking during consumption. However, the shelf life of tortillas is greater than bread, as tortillas retain their protein functionality and have decreased starch dispersion and firming as compared to bread. The diameter of tortillas has extensible dough that resists shrinking back during processing. The dough extensibility in-turn depends again on the gluten proteins and their interactions to form the gluten network. Thus, the dough extensibility during hot pressing and retention of tortilla flexibility after baking have a gluten functionality that is unique to the strong viscoelastic gluten functionality used for bread.

In some embodiments, one uses bread wheat flours and adds various reducing agents to mask the strong bread gluten for increasing the diameter, extensibility and shelf stability of tortillas. L-cysteine is widely used as a reducing agent. It competes with the disulfide bridge-forming cysteine residues in the gluten matrix. The addition of these compounds may negatively affect the taste and quality of tortillas.

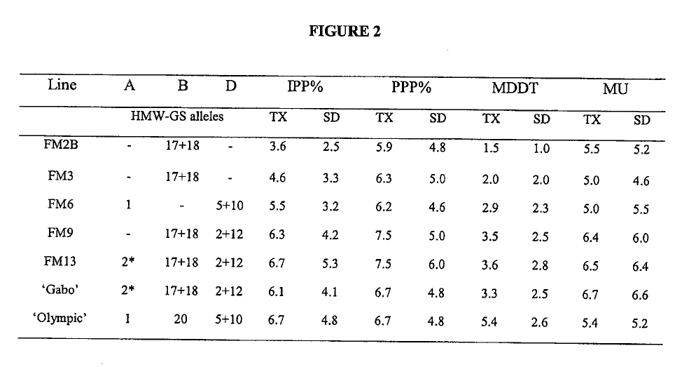

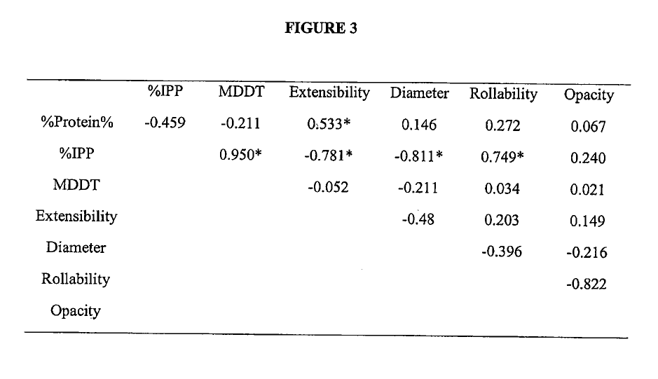

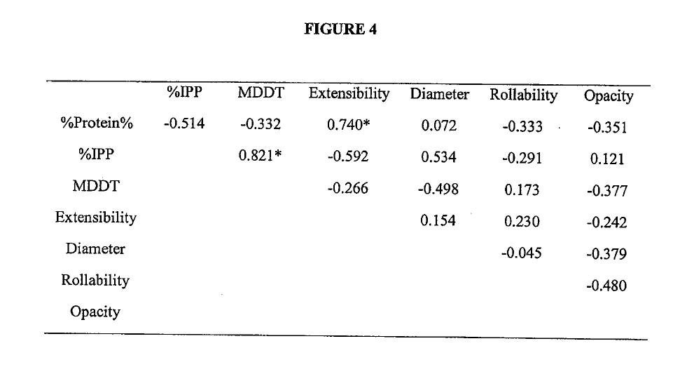

The effect of the HMW glutenins on tortilla quality is evident from the results obtained. The flour protein content in Texas was higher by almost 2% from South Dakota. The higher temperatures in Texas may have increased the protein accumulation via suppression of starch accumulation. The deletion lines had decreased protein content in South Dakota except the lines Fm6 and ‘Gabo’ that had an increase in protein content in South Dakota. While the protein content increased, the % IPP in Fm 6 and ‘Gabo’ had a decrease similar to the other lines in South Dakota (FIG. 2). The lower protein contents reduced the mixographs strength for all lines when grown in South Dakota. The effects on the dough mixing strength due to the specific subunit composition of the HMW glutenin in the deletions were also significant and independent of protein content in some cases. The parent ‘Gabo’ has HMW-GS subunits 2*, 17+18 and 2+12 at GluA1, GluB1 and GluD1 and had higher % IPP values and a strong dough (FIG. 2) even with 2+12, which is associated with weak dough strength. The line Fm9 has the same subunits at GluB1 and GluD1, but a deletion in GluA1, had % IPP almost similar to ‘Gabo’ and a strong mixing strength (FIG. 2). Thus, 17+18 and 2+12 together can give rise to stronger dough mixing strengths. The line Fm6, which has subunits 1 and 5+10 at Glu A1 and GluD1, respectively and a deletion in Glu B1, had a significantly lower % IPP than Fm9 and ‘Gabo’ and had an intermediate dough strength. The lines Fm2B and Fm3 have subunit 17+18 only at GluB1 and deletions at other loci. These two lines showed lower % IPP than Fm6, Fm9 and ‘Gabo’ and a reduced dough mixing time. Thus, the subunits at GluA1 and GluD1 are important in contributing to greater dough mixing strength. The strong correlations with the % IPP and the dough mixing time support the above statement (FIGS. 3 and 4).

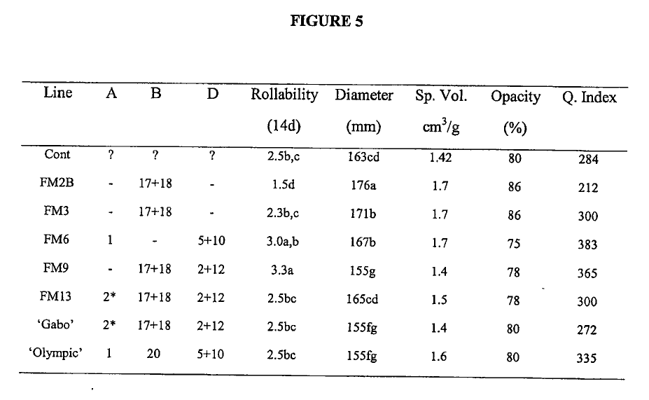

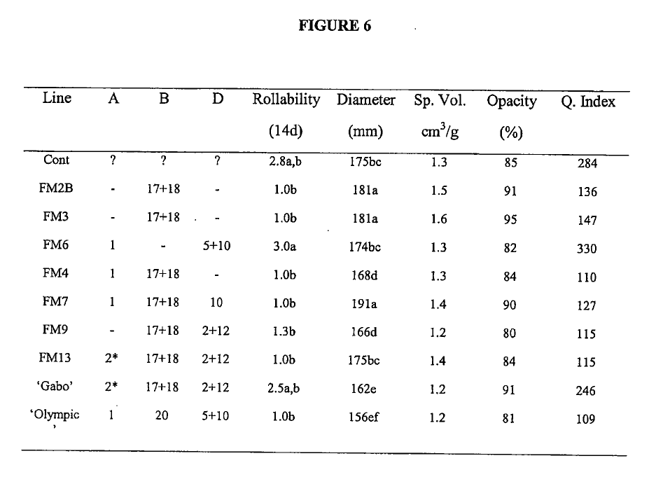

The HMW glutenin functionality also altered specific tortilla properties. Since cysteine was not used in any of these experiments the tortilla properties were due to the functionality of the glutenins present in the flour. Deletion of specific HMW glutenin loci affected unique aspects of the tortilla quality. Tortillas made from lines Fm2B and Fm3 have larger diameters in both Texas and South Dakota (FIGS. 5 and 6, respectively). The diameters are nearly 1-2 cm larger than the control tortilla flour and the parent cultivars. Fm2B has subunits 17+18 at GluB1 and poor rollability scores in both locations. Fm3 had a better rollability score in Texas that was statistically equivalent to the parents. Tortillas made from line Fm6 with subunits 1 and 5+10 and a deletion in GluB1 had better rollabilities on d14 and also significantly (p<0.05) larger diameters compared to the control flour and the parent cultivars, though less than the diameters of Fm2B and FM3. Line Fm 4 tortillas with HMW-GS 1 and 17+18 had smaller diameters as well as a poor rollability (FIG. 6). In this line the interactions of subunit 1 from GluA1 with 17+18 on GluB1 altered the diameter versus Fm3 and Fm2B with only 17+18. The absence of the GluD1 locus also had a negative effect on the rollability of the tortillas. The line Fm 7 which has 1, 17+18 and 10 had a larger diameter than most of the lines yet the rollabilities were poor. Thus while the absence of subunits 5+10 of Glu D1 yielded larger diameter tortillas, the rollabilities were nonetheless lowered. Therefore subunit 5+10 appears important for tortilla shelf stability. The line Fm 9 which has 17+18, 2+12 and a deletion in GluA1 had small diameters. The lines Fm 13 and ‘Gabo’ had a similar composition of 2*, 17+18 and 2+12 and produced tortillas with small diameters and poor rollabilities. The interactions between the subunits 2*, 17+18 and 2+12 did not seem to contribute to good tortilla diameter. Though similar in composition Fm13 and ‘Gabo’ had differences between their diameters and rollabilities. The HMW glutenin compositions are not able to account for all the discrepancies in the lines and the changes may have been due to other reasons such as variations in LMW-GS and gliadin alleles or starch composition. However, the presence of GluD1 HMW-GS subunits does confer a gain in function in tortilla rollability. The HMW-GS subunits at GluB1 alone do not confer good rollabilities, yet when combined with the GluD1 better stability is observed. Tortillas possessing subunits 5+10 had better rollability scores than subunits 2+12.

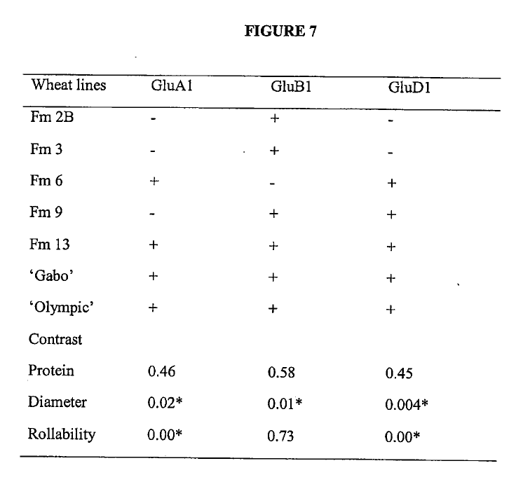

A contrast between the glutenin subunits supports the results obtained for the effects of the subunit composition on the tortilla quality parameters (FIG. 7). Deletions in GluA1 significantly affected the diameter and rollability of the tortillas while not affecting the flour protein content. Deletions in GluB1 loci also improved the diameter significantly with no significant effect on rollability and flour protein content. Deletions in GluD1 loci significantly affected the diameter and the rollability of the tortillas while also not altering the flour protein content. Tortillas prepared from Fm2B and Fm3 with deletions in the GluA1 and GluD1 HMW-GS subunits had significantly larger diameters and lower shelf stability. The tortillas prepared from Fm6 with deletion in the GluB1 HMW-GS had slightly smaller diameters than Fm2B and Fm3 yet had good shelf stability. Thus the interaction between GluA1 and GluD1 HMW-GS or GluD1 HMW-GS alone appears to be a factor to consider for shelf stability.

The % IPP also supports the effects of the glutenin subunit composition on tortilla properties. The deletion of GluA1 and GluD1 reduced the dough mixing strength significantly (FIG. 2). The reduction in the HMW glutenins in these two lines also resulted in increased extensibility of the dough. The HMW/LMW ratio reduced and thus the polymer network formed was weaker as is evident from the dough mixing strength. The reduced amount of specific glutenins forms a weak network able to extend, yet the weak structure reduces its stability and the network ruptures quickly. A lack of polymer forming glutenin also reduced the elasticity of the network. The deletion lines Fm2B and Fm3 were thus able to extend but could not maintain their stability. In Texas the % IPP were higher, hence the stability of the network was better than lines grown in S. Dakota. The lines Fm13, ‘Gabo’ and ‘Olympic’ had higher % IPP. The proportions of HMW glutenins were higher because of the presence of all the glutenin loci GluA1, GluB1 and GluD1. ‘Olympic’ has an intermediate dough mixing strength due to subunit 20 present in GluB1, which may contribute less to the strength than subunit 17+18. This complex network resulted in increased elasticity of the network. The stability of the network was good initially but it dropped by day 14. The lines missing GluB1 had an intermediate dough strength and the % IPP were also moderate. The HMW glutenin network formed was mellow with good extensibility. The stability of the network was also good. Thus, better quality tortillas with a combination of bigger diameters and longer shelf stability can be obtained with moderate % IPP that forms a mellow gluten network with intermediate dough strength.

As such, the line Fm6 had the best overall tortilla quality attributes when compared to the other deletion line. The absence of the subunit 17+18 at the GluB1 locus resulted in gain of function in this line in terms of stability and diameter. The other lines such as Fm2B and Fm3 also had good dough extensibility and tortilla diameters attributes. However, the shelf life of these tortillas was poor, though not different from the control flour and parent lines. The introgression of the deletion compositions possessing only GluB1 HMW-GS into a more adapted background may, however, help compensate for the poor shelf stability. Fm6, among all of the deletion lines, parent lines and the other lines included in the comparison, had the best combined compromise of greater diameter with a longer shelf life. It was better in quality than the commercial tortilla flour possessing L-cysteine for improved extensibility. Fm6 performed better in both locations. This line has a subunit composition of 1 and 5+10 at GluA1 and GluD1 and has potential as a line with better tortilla quality attributes. It also has acceptable loaf volume and may be compatible in a hard red winter wheat distribution system that targets bread quality. The HMW-GS composition found in Fm2B and Fm3, which has very good dough properties and diameter attributes, could be used in tortilla mixes. These tortilla mixes are usually used to make tortillas that are eaten fresh. The tortillas from these lines were fluffier and whiter in color and would be preferred by consumers based on their appearance and light texture. Small businesses and households would appreciate the ease of mixing and the dough processing attributes that these lines possess.

### Gliadins

Based on their electrophoretic mobilities, the gliadins are classified into four different groups: α, β, γ, and ω-gliadins. The genes encoding gliadin proteins are located on short arm of chromosomes 1 and 6. The Gli1 loci has tightly linked genes located at the three homologous loci on the short arm of Chromosome 1, GliA1, GliB1, and GliD1, and in short arm of Chromosome 6, GliA2, GliB2, GliD2 for Gli2 loci. The ω, γ gliadins encoded at the Gli1 loci are tightly linked to the LMW glutenins. The α, β gliadins are encoded by the Gli2 loci.

Polyproline/glutamine tracts are poor substrates for gastrointestinal (GI) endoproteases, such as those produced in the GI tract. People with gluten-sensitive enteropathy (the severe form of which is celiac disease) are sensitive to α, β, and γ gliadins. Those with wheat-dependent (WD) exercise-induced anaphylaxis, WD urticaria and Baker's asthma are sensitive to ω-gliadins.

One example of a gliadin resistant to proteases is a 33-mer of α-2 gliadin LQLQPFPQPQLPYPQPQLPYPQPQLPYPQPQPF (SEQ ID No.: 1) (residues 57 to 89). Three distinct patient-specific T cell epitopes are present in this peptide, namely,

| | |
|---|---|
| PFPQPQLPY, | (SEQ ID No.:2) |
| PQPQLPYPQ, | (SEQ ID No.:3) |
| and | |
| PYPQPQLPY. | (SEQ ID No.:4) |

Another digestion resistant region is a 25-mer of α-gliadin which contains the innate peptide, the 25-mer LGQQQPFPPQQPYPQPQPFPSQQPY (SEQ ID No.: 5), residues 31-55.

### Malting

Glutens in grasses are storage proteins that are designed to help the plant grow during its early life, and among the plant proteins are enzymes that convert starch to sugar. These proteins are activated during sprouting and the starch around the endosperm is converted to sugars, later the prolamins are broken down to provide the young seeds with a source of nitrogen and energy. Once the starch is converted to sugar it can be readily fermented by, e.g., Saccharomyces cerevisiae; however, first the sprouting process should be stopped. In order to do this the partially sprouted grains are placed in a roasting oven and roasted until the sprouts are sterilized and dried, this process of sprouting and drying is called malting. Then the roasted sprouts are ground, rehydrated and fermented, producing a crude beer.

### EXPERIMENTAL

The following examples are provided in order to demonstrate and further illustrate certain preferred embodiments and aspects of the present invention and is not to be construed as limiting the scope thereof.

As used herein, “Glu” refers to Glutenins. As used herein, “HMW-GS” refers to High Molecular Weight Glutenins. As used herein, “IPP” refers to Insoluble Polymeric Proteins. As used herein, “PPP” refers to Polymeric Protein Percent.

### Example I

### Plant Material and Growth Conditions

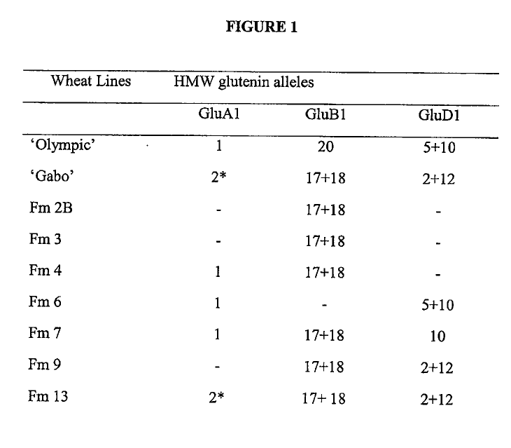

The near-isogenic deletion lines were developed from mutant lines of the wheat cultivar ‘Olympic’, null at GluB1 locus, and an isogenic line of the cultivar ‘Gabo’, null at GluA1 and GluD1 loci. A set from this series of deletion lines was obtained from Dr. Finlay MacRitchie (Kansas State University, Kansas) (FIG. 1).

The wheat lines were grown in field plots at the Texas Agricultural Experiment Station at College Station and at McGregor, Tex., in 2005. The lines were also grown in South Dakota by Dr. Karl Glover, South Dakota State University, Brookings, South Dakota. The parent cultivars ‘Gabo’ and ‘Olympic’ were also grown along with the set of deletion lines.

Performances of these lines in both the locations were evaluated for protein composition and tortilla making ability. Lab-on-a-chip capillary electrophoresis was used to verify the HMW glutenin allelic composition of the field grown deletion lines using the Agilent 2100 Bioanalyzer (Agilent Technologies, Palo Alto, Calif.) as described in Seetharaman et al., J. Cereal Sci. 35, 215-223 (2002), incorporated herein by reference.

### Example II

### Polymeric Protein Analysis

100 mg of flour was extracted with 1 ml 50% aqueous 1-propanol and pellets were freeze-dried before protein determination (N×5.7). Equal volumes of first and second extracts were pooled and analyzed by size exclusion HPLC using a Biosep SEC-4000 column (Phenomenex, Torrance, Calif.) on an Agilent 1100 HPLC system. Column temperature was maintained at 40° C. and the mobile phase was 50% acetonitrile and 0.1% (w/v) trifluoroacetic acid at a flow rate of 0.5 ml/min. Injection volume was 20 ml and UV-detection was done at 210 nm. The percent of insoluble polymeric proteins (IPP) was calculated from the weight and protein content of the freeze-dried pellet, extractable proteins (EP) from the difference between flour protein and protein in the pellet. The allelic composition of the individual near-isogenic deletion lines used in this study is presented in FIG. 2.

The effect of the HMW glutenins on tortilla quality is evident from the results obtained. The flour protein content in Texas was higher by almost 2% from South Dakota. The higher temperatures in Texas may have increased the protein accumulation via suppression of starch accumulation. The deletion lines had decreased protein content in South Dakota except the lines Fm6 and ‘Gabo’ that had an increase in protein content in South Dakota. The lower protein contents also reduced the mixograph strength for all lines when grown in South Dakota. Both the mixograph dough development time (MDDT) and peak dough resistance (MU) were used to describe the strength of the flours. The effects on the dough mixing strength due to the specific subunit composition of the HMW glutenin in the deletions were also significant and independent of protein content in some cases. The parent ‘Gabo’ has HMW-GS subunits 2*, 17+18 and 2+12 at GluA1, GluB1 and GluD1 and had higher % IPP values and a strong dough (FIG. 2) even with subunit 2+12, which is associated with weak dough strength.

### Example III

### Evaluation of Wheat Grain and Flour

A 300-kernel sample was used for determining kernel hardness, diameter, weight and moisture content using the single kernel hardness test (SKHT) (Perten Single Kernel Characterization System SKCS 4100, Perten Instruments, Springfield Ill.). Cleaned grain was tempered to 14% moisture, allowed to rest and milled to flour (Brabender Instruments, South Hackensack, N.J.). Near-infrared reflectance spectrophotometry (NIR) was used to estimate the flour protein content and moisture content from the deletion and parent lines in three separate replicates (Perten PDA 7000 Dual Array with Grams Software). A 35 g sample of flour from each line was used for mixograph analysis to determine the dough mixing time and the dough strength of the flour (Lincoln Manufacturing Company, Lincoln, Nebr.). The dough mixing resistance (MU) and the dough mixing time (MDDT) were recorded from mixograms using standard procedure.

The single kernel hardness test (SKHT) was used to determine the kernel hardness of the lines grown in Texas and South Dakota. Grain hardness indices of 60 and above represent hard grains, while those below 40 are soft grains. ‘Olympic’ and Fm3 are soft with a grain hardness index less than 40. The other lines, Fm2B, Fm 6, ‘Gabo’, and Fm 9, had grain hardness indices above 60 in both locations (not shown). The hardness gene is located in the Chromosome 5B in wheat. In the absence of replicated data a combined analysis was performed for the kernel hardness in two locations. Fishers LSD (LSD=4.31) revealed significant differences between the genotypes for hardness. Fm2B and Fm 3 have similar deletions, yet Fm2B was much harder than Fm3. While deletions in glutenin loci do not affect the kernel hardness, Fm3 may have inherited the hardness gene from ‘Olympic’.

### Example IV

### Dough Evaluation

The dough quality properties were evaluated subjectively. The dough was placed on a plastic tray and the temperature was measured using a thermometer while the values were recorded. The other dough properties such as softness, smoothness, extensibility and force to extend were evaluated subjectively. Smoothness refers to the appearance and texture of the dough rated from 1 to 5, where 1 is very smooth and 5 is rough. The ideal smoothness rating is 2.0. Softness refers to the firmness of the dough when compressed by hand. It is rated from 1 to 5, 1 being very soft and 5 being very firm. The ideal softness rating is 2.0. Extensibility refers to the length to which the dough extends when pulled apart. It was rated from 1 to 5, 1 implying that it breaks immediately and 5 implying that it extends readily. The ideal extensibility is 3.0. Force to extend measures the elasticity of the dough. It is rated from 1 to 5, 1 is less force required and 5 is extreme force required.

The deletions in the glutenin loci resulted in significantly (α=0.05) reduced insoluble polymeric protein content (IPP) (FIG. 2) in Texas (Tukey HSD=0.06) and South Dakota (Tukey HSD=0.11). Even though the protein content increased or stayed the same for Fm6 and ‘Gabo’, the % IPP decreased similar to the other lines in South Dakota (FIG. 2). The line Fm 9, with the same subunits at GluB1 and GluD1 as ‘Gabo’ but a deletion in GluA1, had % IPP almost similar to ‘Gabo’ and a strong mixing strength (FIG. 2). Thus, subunits 17+18 and 2+12 together can give rise to stronger dough mixing strengths. The line Fm6, which has subunits 1 and 5+10 at GluA1 and GluD1, respectively, and a deletion in GluB1, had a significantly lower % IPP than Fm9 and ‘Gabo’ and had intermediate dough strength. The lines Fm2B and Fm3 have subunit 17+18 only at GluB1 and deletions at other loci. These two lines showed significantly lower % IPP than Fm6, Fm9 and ‘Gabo’ and a reduced dough mixing time. Thus, the subunits at GluA1 and GluD1 are important in contributing to greater dough mixing strength. The strong correlations with the % IPP and the dough mixing time support the above statement (FIG. 3).

Subjective dough quality evaluations were also performed. The extensibility of dough without breaking is a parameter that influences tortilla quality, where 3.0 on a scale of 1 (low) to 5 (high) is ideal. Doughs prepared from glutenin deletion lines Fm2B and Fm3 had high dough extensibility scores of 4.5 and 3.5 in Texas and South Dakota respectively (not shown). The other lines had extensibility scores of 3.0 that are near ideal. The elasticity scores indicate the force needed to extend, where 2.0 is ideal for tortillas on scale of 1 (low) to 5 (high). Doughs prepared from lines Fm2B and Fm3 had ideal elasticity scores of 2.0 (not shown). The dough extensibility score of line Fm6 was 2.3. The other lines had higher elasticity scores of 3.0; higher elasticity scores indicate a greater force requirement for extending the doughs. The low elasticity scores with high extensibility scores indicated that these lines had doughs that had good extensibilities without breaking and required less force to extend with little shrink-back. The commercially available tortilla control flour also had a good extensibility score but higher elasticity scores of 3.5 and 3.0 respectively (not shown). Good extensibility with high elasticity indicates the nature of the dough to extend and then subsequently shrink from elasticity, resulting in a small diameter tortilla.

The ideal dough smoothness score is 2.0 on a scale of 1 (very smooth) to 5 (highly rough). The lines Fm2B and Fm3 had an ideal smoothness score of 2.0 from both locations. The doughs prepared from other lines grown in both locations had a smoothness score above the ideal score. The dough prepared from the lines Fm2B and Fm3 had softness scores of 2.5 and 2, respectively, in both locations, indicating these lines produce softer doughs. Doughs from the other lines as well as the control flour were firmer.

Significant correlations were observed between the force to extend and the protein content (FIG. 3). A significant correlation of 0.533 (p<0.05) was observed in Texas. The increased protein content increased the force required to extend the dough. Dough extensibility was also significantly correlated (p<0.05) with flour protein content.

### Example V

### Tortilla Processing and Evaluation

The flour from each line grown in Texas and South Dakota were processed into tortillas. The tortillas were prepared according to a standard formulation except that cysteine was not added. The formulation was standardized as 500 g of flour, 7.5 g of salt, 2.5 g of sodium stearoyl lactylate, 2 g of potassium sorbate, 2.3 g of encapsulated fumaric acid, and 30 g of shortening. The amount of water added was based on mixographs of the water absorption. Commercially available tortilla flour (ADM Tortilla Flour, ADM Milling Company, Overland Park, Kans.) was used to compare the tortilla quality obtained from the commercial flour and the selected experimental lines. The tortillas from each experimental line were made in two batches, a smaller amount of flour to standardize the formulation and water requirement, and a second batch made from 500 g of flour used for evaluation.

Dry ingredients were mixed with the flour in a mixing bowl with a paddle at a low speed for 1 min placed over copper tubes through which heated water at 70° C. was pumped to control temperature (Model A-200, Hobart Corporation, Troy, Ohio). Shortening was then added and paddle mixed for 2 min at low speed. Water (35° C.) was then added and mixed for 1 min at low speed and then mixed at a medium speed for 6 min with a hook.

The dough was placed in a plastic tray for dough quality measurements. The dough was then proofed (model 57638, National Manufacturing Company, Lincoln, Nebr.) at 35° C. and 70% relative humidity for 5 min. The dough was pressed by hand and divided and rounded with Duchess Divider/Rounder (Bakery Equipment and Service Company, San Antonio, Tex.) into 36 dough balls of 43 g each. The dough balls were transferred to the plastic tray and rested in the proof chamber for 10 min at 35° C. and 65% relative humidity.

The dough balls were placed on a hot press (Micro-Combo model 0P01004-02, Lawrence Equipment Company Incorporation, South El Monte, Calif.) and pressed at 1100 psi. The tortillas were then baked in the three-tier oven (Micro-Combo Tortilla Oven, Model 0P01004-02, Lawrence Equipment, South El Monte, Calif.) set at a temperature of 350-360° F. The dwell time was adjusted to 30 sec. The tortillas were cooled on a three-tier cooling chain (model 3106 INF, Food Machinery Incorporation/Pivo Machinery Incorporation, Pico Rivera, Calif.), removed placed on a table for 1 min, flipped on the other side for cooling and packed in low-density polyethylene bags and stored at 23° C. for quality evaluation.

The tortillas were evaluated for their weight, diameter, height, pH, moisture, opacity, color and rollability. Using a balance, ruler and digital caliper, respectively, the weights, diameters from two points, and height from 10 individual tortillas were averaged. The pH and moisture content of individual tortillas from each line was determined.

The opacity of 10 tortillas was subjectively evaluated using a continuous scale of 1-100% (1% being fully translucent and 100% being high opacity). The values were recorded and averaged. The color parameters, L*(lightness), ±a*(red-green), and ±b*(yellow-blue) were measured for each tortilla using a Minolta Color Meter (Chroma Meter CR-310, Minolta, Tokyo, Japan) using three measurements on each side of the tortillas. Tortilla shelf stability was evaluated using the rollability test by wrapping a tortilla around a wooden dowel (1.0 cm in diameter). Ratings on a scale of 1 to 5 were recorded with 1 being immediate breakage and 5 being no cracks or breakage. The rollabilities were evaluated on the 4^{th}, 10^{th} and 14^{th} days following processing for each of the lines. Three tortillas from each of the lines were used for the measurements. The specific volume was then calculated for each of the lines (cm³/g). The specific volume indicates the fluffiness of the tortillas. It ranges from 1.5-3.5 cm³/g. The specific volume was calculated by the formula:

π*(Diameter/2)2*height*1000/weight.

The quality index was then calculated based on the opacity, rollability and specific volume by using the formula:

Opacity*Specific volume*Rollability score (14^{th} day of rollability).

The HMW glutenin functionality also altered specific tortilla properties. Since cysteine was not used in any of these experiments and the protein content was statistically unchanged the tortilla properties were due to the functionality of the glutenins present in the flour and/or their influence on the insoluble polymeric protein fraction. Deletion of specific HMW glutenin loci affected unique aspects of the tortilla quality. The control flour tortillas had a diameter of 163 mm, significantly larger than tortillas prepared from both parent cultivars “Olympic” and “Gabo” (155 mm) (FIG. 1). In Texas, with the exception of Fm9, all deletion lines had significantly larger tortilla diameters than the parent lines (FIG. 1). Tortillas prepared from deletion lines Fm2B and Fm3 were exceptional at 176 and 171 mm in diameter, respectively, which was 1-2 cm larger than tortillas prepared from the control tortilla flour, the parent cultivars, and other lines. Tortilla prepared from Fm6 and Fm13 (167 mm and 165 mm in diameter, respectively) were also larger than the control flour and parent cultivars (significant, p<0.05). The South Dakota tortilla diameter evaluations supported the Texas results. The tortillas prepared from the lines Fm2B, Fm 3 and Fm7 were the largest (181 mm, 181 mm and 191 mm, respectively), lines Fm6 and Fm13 were intermediate (174 mm and 175 mm, respectively), and each was significantly larger than the control flour and parent lines ‘Gabo’ and ‘Olympic’ (FIG. 1).

The line Fm2B has subunits 17+18 at GluB1 and poor rollability scores in both the locations. Fm3 had a better rollability score in Texas that was statistically equivalent to the parents. Tortillas made from line Fm6 with subunits 1 and 5+10 and a deletion in GluB1 had better rollabilities on day 14 and also significantly (p<0.05) larger diameters compared to the control flour and the parent cultivars, though less than the diameters of Fm2B and Fm3. Line Fm 4 tortillas with HMW-GS subunits 1 and 17+18 had smaller diameters as well as a poor rollability (FIG. 2). In this line the interactions of subunit 1 from GluA1 with 17+18 on GluB1 altered the diameter versus Fm3 and Fm2B with only 17+18. The absence of the GluD1 loci also had a negative effect on the rollability of the tortillas. The line Fm7, which has subunits 1, 17+18 and 10, had a larger diameter than most of the lines, yet the rollabilities were poor with breakage by day 14. Thus, while the absence of subunit 5+10 of GluD1 yielded larger diameter tortillas, the rollabilities were nonetheless lowered. Therefore, subunit 5+10 appears important for tortilla shelf stability. The line Fm9, which has 17+18, 2+12 and a deletion in GluA1, had small diameters. The lines Fm13 and ‘Gabo’ had a similar composition of subunits 2*, 17+18 and 2+12 and produced tortillas with small diameters and poor rollabilities. The interactions between the subunits 2*, 17+18 and 2+12 did not seem to contribute to good tortilla diameter. Though similar in composition, Fm13 and ‘Gabo’ had differences between their diameters and rollabilities. The HMW glutenin compositions are not able to account for all the discrepancies in the lines and the changes may have been due to other reasons such as variations in LMW-GS and gliadin alleles or starch composition. However, the presence of GluD1 HMW-GS subunits does confer a gain in function in tortilla rollability. The HMW-GS subunits at GluB1 alone do not confer good rollabilities, yet when they are combined with the GluD1 better stability is observed. Tortillas possessing subunit 5+10 had better rollability scores than subunit 2+12. This could have been more thoroughly explained if a deletion line containing both the GluB1 17+18 and GluD1 5+10 subunits was available for comparison.

Significant tortillas opacities (p<0.05) differences between the wheat lines were observed. The opacity scores of the tortillas prepared from the glutenin deletion lines were higher than the control flour and the other lines. The ideal opacity scores are above 85. The tortillas prepared from the glutenin deletion lines Fm2B and Fm3 had opacity scores of 86, while the tortillas prepared from other deletion lines and the other varieties had opacity scores similar to the control flour <80 (FIG. 5). Tortillas from glutenin deletion lines with greater diameters had better opacities (FIG. 1).

The rollability scores had a significant genotypic effect in both locations (not shown). The control flour tortilla had a rollability score of 2.5 on day 14 (FIG. 2). Tortillas with rollability scores of 3.0 and above on day 14 have a good shelf life. The parent cultivars ‘Gabo’ and ‘Olympic’ had rollability scores of 2.5 on day 14. A rollability score of the tortillas prepared from the glutenin deletion lines Fm6 and Fm9 had a significantly better shelf stability of 3.0 on day 14. Tortillas from lines Fm2B and Fm3 had lower rollability scores of 1.5 and 2.3, respectively, on day 14 (FIG. 2), though Fm3 was statistically equivalent to tortillas from the control flour and parent lines. The tortillas prepared from other lines had poor rollability scores of 1.5-2. In general, all of the lines from South Dakota had poor rollability scores. The only exceptions were tortillas prepared from the line Fm6 and the parent cultivar ‘Gabo’, which had a significantly higher shelf life and rollability scores of 3.0 and 2.5 on day 14, respectively (FIG. 2).

The quality index calculations were based on the day 14 rollability scores, where a score above 300 is considered good. Some of the lines had a very good quality index in Texas (FIG. 4). The control flour tortilla had a good quality index of 284, with the parent ‘Olympic’ higher at 335 and ‘Gabo’ lower at 272. All deletion lines had quality scores above 300, with Fm6 having the best with a quality score of 383. The only exception was Fm2B, which had a lower quality index due to the low rollability scores on day 14 (FIG. 4). Due to the poor rollability scores in South Dakota, the quality indices were also low. The quality indices from Fm6 and ‘Gabo’ were with higher scores due to better rollability scores. The quality index of Fm6 was better than the control flour in both Texas and South Dakota.

Correlations were calculated between the tortilla quality parameters separately in both the locations using SPSS software (FIG. 3). In Texas tortilla diameters had a strong negative correlation (p<0.05) with the % IPP, while shelf stability (rollability at day 14) was strongly correlated with % IPP (p<0.05) (FIG. 3). In South Dakota the dough extensibility and the protein content had a significant correlation (p<0.05) (FIG. 3).

### Example VI

### Gliadin Alleles

In the Russian cultivar Saratovskaja (Sarat), mutant deletion lines were used. This cultivar was selected as the plant material for the study of the effect of gliadins functionality in tortilla quality. The mutant lines have Sarat with deletions in their Gli1 and Gli2 loci, respectively. These selected gliadin deletion lines and their parent cultivar Sarat were grown in South Dakota.

Polymerase chain reactions were performed with the SSR primer pair Xgwm147 located near the Gli1 locus in short arm of Chromosome 1A. A deletion in the short arm of Chromosome 1 containing the GliA1 locus in the gliadin deletion line GliA1 locus resulted in no amplification with the primer Xgwm147. The other lines, GliA2, GliD1, GliD2 and parent cultivar Sarat, had bands in the agarose gels, verifying the presence of the GliA1 locus. PCR was similarly performed to verify the deletions in the Gli1 and Gli2 loci present in the gliadin deletion lines GliA2, GliD1 and GliD2 with the SSR primers pairs Xgwm459, Xgwm106 and Xgwm469 respectively. The PCR results were further verified using the HEPC analysis.

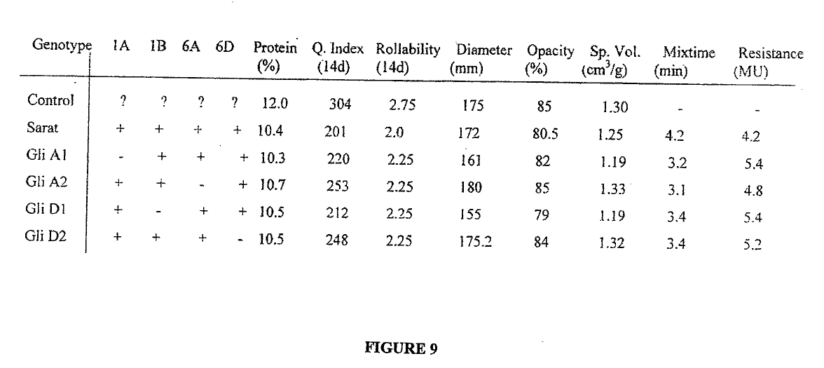

The HEPC analyses performed supported the results from the PCR analysis. The HEPC analysis verified the presence of gliadin deletions in the line GliA1, GliA2, GliD1 and GliD2 loci. In FIG. 9 the deletions in the gliadin deletion lines are demonstrated with respect to the parent line Sarat. Absences of some peaks indicate the deletions. Line GliA2 had some of α- and β-gliadin peaks absent. In the line GliD2 co-gliadin peaks are absent, indicating the deletions in Gli2 locus.

The extraction of the flour proteins reveals that the polymeric protein percent was increased in the lines with gliadin deletions. This was expected as the reduction in the monomeric proteins corresponded with an increase in the polymeric proteins and an increase in the glutenin to gliadin ratio. The Tukey HSD analysis confirmed the significant increase in % IPP in the gliadin deletions lines compared to the parent cultivar. The only exception was the line GliA2 that has similar % IPP as the parent cultivar. FIG. 9 contains the deletions present and the changes in the % IPP and dough mixing time. The deletions in gliadin alleles have significantly affected the % IPP as obtained from the contrasts.

The flour protein content from NIR of the parent cultivar was 10.4%. The gliadin deletion lines GliA1, GliA2, GliD1 and GliD2 had flour protein content similar to the parent line of around 10.5%.

The dough quality evaluation demonstrates that the extensibility of the dough is increased even when there is reduction in the monomeric proteins. It is quite contrary to the earlier studies. In all of the gliadin deletion lines an extensibility score of 3.0 was found, similar to the score for the parent Sarat and the control tortilla flour. The elasticity scores are above the ideal score of 2.0. The doughs were extensible even though there was an increase in the % IPP. Except for the lines GliA2 and GliD2 all of the lines had an ideal dough softness rating of 2.0. The gliadin deletion lines GliA2 and GliD2 had very soft doughs.

The gliadin lines behaved opposite to our expectations. The deletions in gliadin loci increased the polymeric proteins. The increase in the polymeric proteins should decrease the tortilla diameter. However, the tortillas prepared from the gliadin deletion line GliA2 and GliD2 had significantly large diameters (p<0.05). The diameters were 180 mm and 175.2 mm, respectively. These tortilla diameters were similar to the diameters obtained from the deletions of the HMW glutenins and larger than the tortilla diameters from the parent cultivar Sarat and the control flour. The tortillas prepared from the gliadin lines GliA1 and GliD1 had smaller diameters as expected due to the increase in their % IPP. The parent line Sarat had a diameter of 172 mm.

The tortillas prepared from the gliadin deletion lines GliA1, GliD1, GliA2 and GliD2 had similarly poor rollability scores on the day 14 (FIG. 9). No significant differences in the tortilla rollability scores were observed between the gliadin deletion lines and the parent and the control flour by the Tukey-HSD test on the 14^{th} day. The rollabilities of all of the deletion lines were within the range of that found for the control flour.

What is claimed is:

1. A composition produced from crushing a wheat grain or portion of a wheat grain, wherein said grain does not express a wild-type gene product from one or more loci encoding a protein selected from the group consisting of glutenins and gliadins.

2. The composition of claim 1, wherein said composition is flour.

3. A dough produced from the flour of claim 2, wherein said dough has a decreased elasticity compared to dough derived from a wheat line that does express said gene product from said loci.

4. The flour of claim 2, wherein said wheat grain does not express said gene product due to a deletion of a chromosome arm, deletion of said gene, or deletion of a portion of said gene at said loci.

5. The flour of claim 2, wherein said wheat does not express said gene product due to a mutation of said gene at said loci.

6. The flour of claim 2, wherein said grain does not express a wild-type gene product from two or more loci encoding a protein selected from the group consisting of glutenins and gliadins.

7. A flat bread produced from said dough of claim 3.

8. The flat bread of claim 7, wherein said flat bread is selected from the group consisting of tortilla, pizza dough, and pita.

9. The flour of claim 1, wherein said wheat grain is obtained from a wheat line that is hexaploid and near-isogenic.

10. The flour of claim 1, wherein said loci selected from the group consisting of GluA1, GluB1, GluD1, GliA1, GliB1, GliD1, and Gli2A.

11. A wheat line comprising a null allele at a GluB1 locus.

12. The wheat line of claim 11, further comprising a null allele at a GluA1 locus.

13. The wheat line of claim 11, further comprising a GluA1 gene that expresses a protein subunit selected form the group consisting of subunit 1 and subunit 2*.

14. The wheat line of claim 11, further comprising a null allele at a GluD1 locus.

15. The wheat line of claim 11, further comprising one or more GluD1 genes that express a protein subunit selected from the group consisting of subunit 2, subunit 3, subunit 4, subunit 5, subunit 10, subunit 11 and subunit 12.

16. The wheat line of claim 11, further comprising a GluD1 gene that expresses protein subunits selected from the group consisting of subunits 5 and 10, subunits 2 and 12, subunits 3 and 12, subunits 4 and 12, subunits 2 and 11.

17. The wheat line of claim 11, wherein said wheat line does not express a gene product from one or more loci selected from the group consisting of GliA1, GliB1, GliD1 and Gli2.

18. A near-isogenic wheat line of claim 11.

19. A wheat line comprising a null allele at a GluB1 locus, a GluA1 gene that expresses a protein subunit 1, and a GluD1 gene that expresses protein subunits 5 and 10.

20. A near-isogenic wheat line of claim 19.

21. A flour made from crushing the wheat grains of the wheat line of claim 19.

22. A wheat line comprising a null allele at a GluA1 locus and GluB1 gene expressing a protein subunit selected form the group consisting of subunit 6, subunit 7, subunit 8, subunit 9, subunit 13, subunit 14, subunit 15, subunit 16, subunit 17, subunit 19, subunit 20, subunit 21 and subunit 22.

23. The wheat line of claim 22, further comprising a GluB1 gene expressing protein subunits selected from the group consisting subunits 6 and 8, subunits 7 and 8, subunits 7 and 9, subunits 14 and 15, and subunits 17 and 18.

24. The wheat line of claim 22, further comprising a null allele at a GluD1 locus.

25. The wheat line of claim 22, further comprising a GluD1 gene expressing a protein subunit selected from the group consisting of subunit 2, subunit 3, subunit 4, subunit 5, subunit 10 and subunit 12.

26. The wheat line of claim 22, comprising a GluD1 gene expressing protein subunits selected from the group consisting of subunits 5 and 10, subunits 2 and 12, subunits 3 and 12, subunits 4 and 12 and subunits 2 and 11.

27. The wheat line of claim 22, wherein said wheat line does not express a gene product from one or more loci selected from the group consisting of GliA1, GliB1, GliD1 and Gli2.

28. A wheat line comprising a null allele at a GluA1 locus, a GluB1 gene expressing protein subunits 17 and 18 and a GluD1 gene expressing subunits 2 and 12.

29. A flour made from crushing a wheat grain of the wheat line of claim 28.

30. A near-isogenic wheat line of claim 28.
